(19) United States
(12) Patent Application Publication
(10) Pub. No.: US 2023/0323269 A1
(43) Pub. Date: Oct. 12, 2023

(54) Sampling Device And Cell Culture System

(71) Applicant: Terumo Kabushiki Kaisha, Tokyo (JP)

(72) Inventor: Masatsugu IGARASHI, Kanagawa (JP)

(21) Appl. No.: 18/206,154

(22) Filed: Jun. 6, 2023

Related U.S. Application Data
(63) Continuation of application No. PCT/JP2022/008729, filed on Mar. 2, 2022.

(30) Foreign Application Priority Data
Mar. 3, 2021 (JP) 2021-033617

(51) Int. Cl. C12M 1/26; C12M 1/00; C12M 1/12; C12M 1/34

(52) U.S. Cl. CPC C12M 33/04; C12M 23/40; C12M 37/02; C12M 41/26; C12M 41/34; C12M 41/36

(57) ABSTRACT

A cell culturing system configured to move a liquid sample from a cell culturing device to a sampling channel via a sample introduction channel. The sampling device includes a sampling channel having a main-mechanism-side pump disposed between a cleaning solution storage unit and the sample introduction channel and is configured to allow a cleaning solution to flow to a detection unit. The sample introduction channel includes an introduction-mechanism-side pump that is configured to allow the sample to flow from the sample introduction channel to the detection unit.

### CROSS-REFERENCE TO RELATED APPLICATIONS

The present application is a continuation application of the International Patent Application No. PCT/JP2022/008729 filed on Mar. 2, 2022, which designated the U.S. and claims the benefit of priority from Japanese Patent Application No. JP2021-033617 filed on Mar. 3, 2021. The entire disclosures of the above-identified applications are incorporated herein by reference.

### FIELD

The present disclosure relates to a sampling device for collecting a liquid sample from a cell culturing device, and a cell culturing system that includes the sampling device and the cell culturing device.

### BACKGROUND

A sampling device for collecting a liquid sample from a culturing device. The sampling device includes a sampling channel and a pump that is configured to draw a sample from a sample introduction channel that is connected to the culturing device and into the sampling channel. The sampling device may also include a detection unit downstream of the sampling channel. The detection unit is configured to detect components contained in the sample and amounts (e.g., concentrations) of the components.

Cells cultured using such culturing device need to be cultured under aseptic conditions, and the sampling device used with and connected to the culturing device should be configured to ensure sterility and aseptic conditions. Often sensors used for the detection unit are unsuitable for sterilization. The sensors are attached to a circuit, and thus, the sterility of the circuits is often also not maintained.

An aseptic filter may be disposed between the sampling device and the culturing device. A pump provided downstream of a sampling flow path may be configured to draw the sample from the sample introduction channel and into the sampling flow path via the aseptic filter. In such instances, the sample introduction channel may have a negative pressure. When the sample introduction channel is closed by a clamp, for example, for cleaning, while the sample introduction channel has a negative pressure, and the clamp then opened, a cleaning solution may flow into (i.e., flows back into) the sample introduction channel. If the negative pressure is too large, the cleaning solution may then flow into the culturing device. Thus, if the sterility of the sampling device is not ensured, the sterility of the culturing device may be compromised.

Accordingly, there is a need for a sampling device and a cell culturing system configured to maintain sterility particularly with respect to a sample introduction channel for introducing a sample and effectively preventing inflow of a cleaning solution from the sampling channel.

### SUMMARY

The present disclosure provides a sampling device for collecting a liquid sample from a cell culturing device In at least one example embodiment, the sampling device may include: a sampling channel through which the sample flows; a detection unit disposed in the sampling channel and configured to contact the sample; a cleaning solution storage unit connected to (e.g., in fluid communication with) the sampling channel upstream of the detection unit and configured to store a cleaning solution. A sample introduction channel may be configured to connect (e.g., to establish fluid communication between) the cell culturing device and the sampling device. For example, the sample introduction channel may connect to (e.g., be in fluid communication with) the sampling channel between the detection unit and the cleaning solution storage unit. the sample introduction channel may enable introduction or movement of the sample from the culturing device into the sampling channel. The sampling device may further include a first pump and a second pump. The first pump may be is provided in the sampling channel between the cleaning solution storage unit and the sample introduction channel and that is configured to allow the cleaning solution to flow to the detection unit. The second pump may be provided in the sample introduction channel and may be configured to allow the sample to flow to the detection unit from the sample introduction channel.

The present disclosure provides a cell culturing system that includes a culturing unit for culturing a cell. The cell culturing system may include a sampling channel through which a liquid sample collected from the culturing unit flows; a detection unit disposed in the sampling channel and configured to contact the sample; a cleaning solution storage unit that is connected to (e.g., in fluid communication with) the sampling channel upstream of the detection unit and configured to store a cleaning solution; and a sample introduction channel connected to (e.g., in fluid communication with) the culturing unit and connected to (e.g., in fluid communication with) the sampling channel between the detection unit and the cleaning solution storage unit. The sample introduction channel may enable introduction or movement of the sample from the culturing unit into the sampling channel. The cell culturing system may include a first pump and a second pump. The first pump may be provided in the sampling channel between the cleaning solution storage unit and the sample introduction channel and may be configured to allow the cleaning solution to flow to the detection unit. The second pump may be provided in the sample introduction channel and may be configured to allow the sample to flow to the detection unit from the sample introduction channel.

The sampling device and the cell culturing system may be configured to prevent entry of the cleaning solution from the sampling channel into the sample introduction channel.

### BRIEF DESCRIPTION OF DRAWINGS

FIG. 1 is a perspective view of a cell culturing system in accordance with at least one example embodiment of the present disclosure.

FIG. 2 is a schematic illustrating a channel for a culture medium in the cell culturing system illustrated in FIG. 1 in accordance with at least one example embodiment of the present disclosure.

FIG. 3 is a schematic illustrating an example channel of a sampling device configured to be used with the cell culturing system illustrated in FIG. 1 in accordance with at least one example embodiment of the present disclosure.

FIG. 4 is a perspective view of the sampling device in accordance with at least one example embodiment of the present disclosure.

FIG. 5 is a perspective view of a first sensor unit and a second sensor unit of the sampling device illustrated in FIG. 4 in accordance with at least one example embodiment of the present disclosure.

FIG. 6 is a flowchart illustrating a sampling method performed by the sampling device illustrated in FIG. 4 in accordance with at least one example embodiment of the present disclosure.

FIG. 7 is a schematic illustrating operations in the priming step and the cleaning step of the sampling method illustrated in FIG. 6 in accordance with at least one example embodiment of the present disclosure.

FIG. 8 is a schematic illustrating operations of the sampling step of the sampling method illustrated in FIG. 6 in accordance with at least one example embodiment of the present disclosure.

FIG. 9 is a schematic illustrating operations of the calibration step of the sampling method illustrated in FIG. 6 in accordance with at least one example embodiment of the present disclosure.

FIG. 10 is a flowchart illustrating a sampling method according to at least one example embodiment of the present disclosure.

FIG. 11 is a schematic illustrating operations of the drawing step of the sampling method illustrated in FIG. 10 in accordance with at least one example embodiment of the present disclosure.

FIG. 12 is a schematic illustrating an example channel of the sampling device configured to be used with the cell culturing system illustrated in FIG. 1 in accordance with at least one example embodiment of the present disclosure.

FIG. 13 is a schematic illustrating an example channel of the sampling device configured to be used with the cell culturing system illustrated in FIG. 1 in accordance with at least one example embodiment of the present disclosure.

FIG. 14 is a schematic illustrating the connection between a cell culturing device and a sampling device that together define a cell culturing systemin accordance with at least one example embodiment of the present disclosure.

### DETAILED DESCRIPTION

Example embodiments of the present disclosure are described in detail below with reference to the drawings.

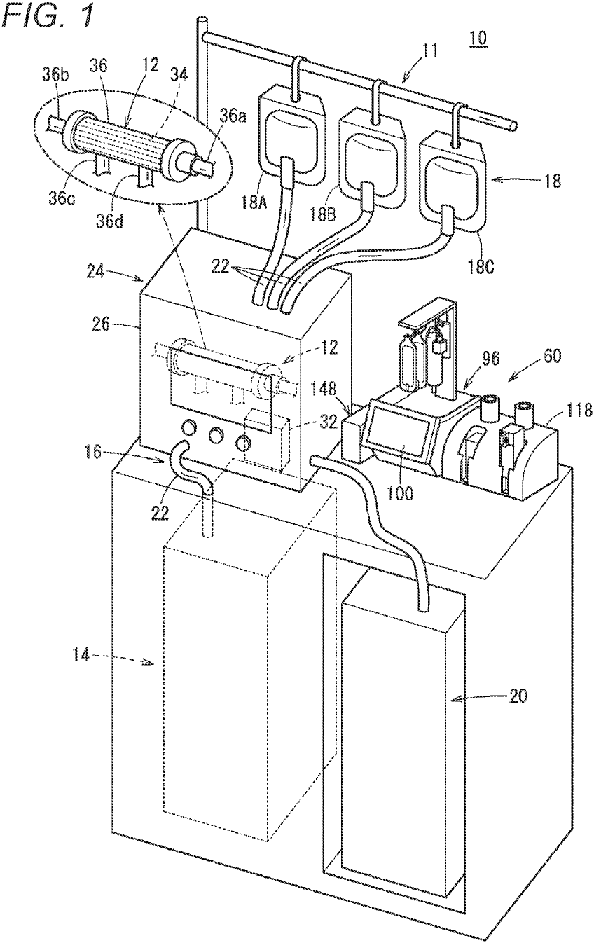

A cell culturing system 10 for culturing biological cells in regenerative medicine is illustrated in FIG. 1. A sampling device 60 may be used with the cell culturing system 10. The sampling device 60 may be configured to sample a culture medium during cell culturing by the cell culturing system 10 and to measure the state of the culture medium. The cell culturing system 10 may continue cell culture for a period of time by discharging lactic acid, carbon dioxide, and/or the like (including unused medium and oxygen) as generated during cell culturing from a reactor 12 (e.g., a cell culture vessel) while supplying a culture medium or oxygen to the reactor 12.

The biological cells for cell culturing are not particularly limited. The biological cells include, for example, cells contained in blood (e.g., T cells and the like) and stem cells (e.g., ES cells, iPS cells, mesenchymal stem cells, etc.). An appropriate culture medium may be selected according to particular biological cells. In at least one example embodiment, the culture medium may be prepared by adding various amino acids, vitamins, serum, and/or the like to a basic solution. The basic solution may include, for example, a balanced salt solution (BSS).

The cell culturing system 10 may include a cell culturing device 11 (which may also be referred to as a culturing unit) in which the reactor 12 is set and cells are actually cultured and the sampling device 60 that collects a liquid sample from the cell culturing device 11. Although the culturing device 11 illustrated in FIG. 1 only has one reactor 12, it should be recognized that in other embodiments, the culturing device 11 may include a plurality of reactors 12. Although in the illustrated embodiment, the culturing unit 11 and the sampling device are separately provided, it should be recognized that in other embodiments, the cell culturing unit 11 and the sampling unit may be integrated(unified).

The culturing device 11 may include a culture medium reservoir 14 configured to store a culture medium, a flow channel 16 provided between the reactor 12 and the culture medium reservoir 14, a plurality of medical bags 18 connected to the flow channel 16, and a waste liquid unit 20 configured to store a liquid discharged through the flow channel 16.

The culture medium reservoir 14 may include a hard tank that is configured to store a large amount of the culture medium. The flow channel 16 may include multiple tubes 22, and the multiple tubes 22 may be connected, respectively, to the reactor 12, the culture medium reservoir 14, the plurality of medical bags 18, and the waste liquid unit 20.

the plurality of medical bags 18 may include a cell solution bag 18A configured to store a liquid containing cells (e.g., cell solution), a cleaning solution bag 18B configured to store a cleaning solution, a stripping solution bag 18C configured to store a stripping solution, and a collection bag (not illustrated) configured to receive cultured cells. The cleaning solution may be a liquid used at the time of priming of the reactor 12 and the flow channel 16. The cleaning solution may include a buffer solution and/or a physiological saline solution. The buffer solution may include, for example, phosphate buffered slats (PBS) and tris-buffered saline (TBS). The stripping solution may be a liquid selected for stripping the cells cultured by a culture treatment. The stripping solution may include, for example, trypsin and/or ethylenediaminetetraacetic acid (EDTA).

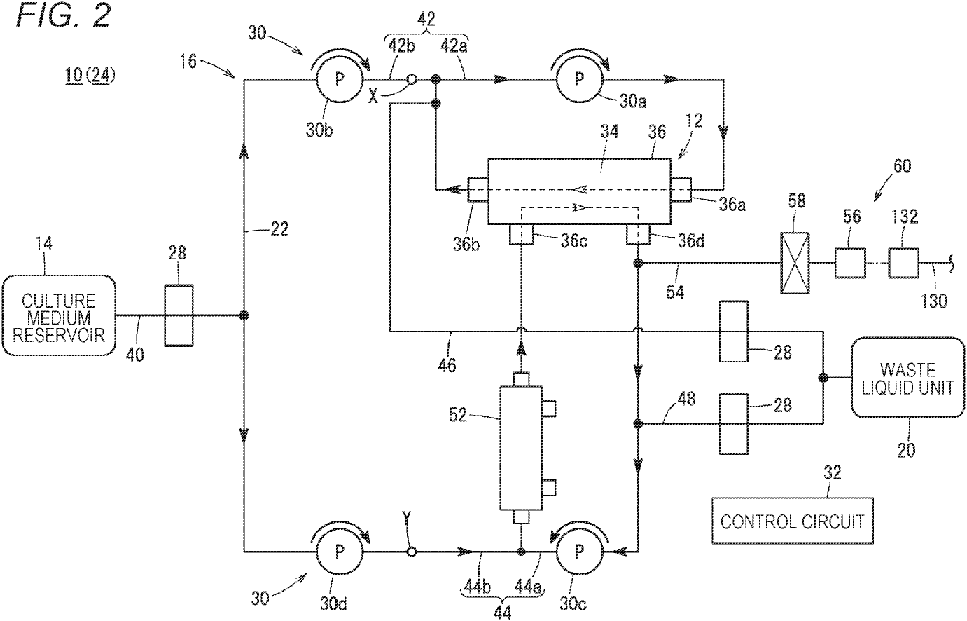

When the cell culturing system 10 is constructed, the flow channel 16 may be set so as to pass through a flow path control mechanism 24 of the culturing device 11. The flow path control mechanism 24 may include a housing 26 that is configured to house a part of the flow channel 16. As illustrated in FIG. 2, the flow path control mechanism 24 may include, in the housing 26, a clamp 28 that is configured to open and close a predetermined tube 22, a pump 30 that is configured to circulate liquid in the tube 22, and/or a control circuit 32 that is configured to control operations of the clamp 28 and the pump 30.

The reactor 12 may also be accommodated in the housing 26. The reactor 12 may include a plurality of (e.g., 10,000 or more) hollow fibers 34 and a case 36 that accommodates the plurality of hollow fibers 34. Each of the hollow fibers 34 may include a lumen (not illustrated), and cells may be seeded on an inner peripheral surface that defines the lumen. Each of the hollow fibers 34 may also have a plurality of pores (not illustrated) that allows communication between the outside and the lumen. The pore may allow for the movement of a solution and/or a low-molecular-weight substance without transmitting cells or proteins. A culture medium or the like may be supplied to the cells seeded on the inner peripheral surface of the hollow fiber 34 through the lumen or the pores. The configuration in which the liquid mainly flows through the lumen of the hollow fiber 34 may be referred to as intra capillary (IC), and the configuration in which the liquid mainly flows through the outer side of the hollow fiber 34 may be referred to as extra capillary (EC) .

The case 36 may include a first IC terminal 36a and a second IC terminal 36b that are connected to the plurality of tubes 22 and communicate with the lumens of the hollow fibers 34. The case 36 may also include a first EC terminal 36c and a second EC terminal 36d that communicate with a space within the case 36 outside of the hollow fibers 34.

As illustrated in FIG. 2, the flow channel 16 may include a medium delivery route 40 connected to the culture medium reservoir 14, and an IC route 42 (internal route) and an EC route 44 (external route) which may be branched from the medium delivery route 40. The IC route 42 may be a channel configured to supply a liquid to the lumen of the hollow fibers 34. The EC route 44 may be a path configured to supply liquid to the case 36 and outside of the hollow fibers 34.

The IC route 42 may include an IC circulation circuit 42a configured to circulate liquid with the reactor 12 and an IC supply circuit 42b through which liquid can flow from the culture medium delivery route 40 to the IC circulation circuit 42a. The IC circulation circuit 42a may be connected to the first IC terminal 36a and the second IC terminal 36b of the reactor 12. The IC circulation circuit 42a may include an IC circulation pump 30a that allows liquid to flow through the lumen of the hollow fibers 34. An IC waste liquid circuit 46 that is configured to discharge a culture medium to the waste liquid unit 20 may be connected to the IC circulation circuit 42a on the downstream side of the reactor 12. In at least one example embodiment, the IC supply circuit 42b may be provided with an IC supply pump 30b configured to allow liquid to flow from the culture medium delivery route 40 to the IC circulation circuit 42a.

In at least one example embodiment, the EC route 44 may include an EC circulation circuit 44a configured to circulate liquid with the reactor 12 and an EC supply circuit 44b through which liquid can flow from the culture medium delivery route 40 to the EC circulation circuit 44a. The EC circulation circuit 44a may be connected to the first EC terminal 36c and the second EC terminal 36d of the reactor 12 and may include an EC circulation pump 30c that is configured to circulate liquid on the outside of the hollow fibers 34. The EC circulation circuit 44a may include a gas exchanger 52 upstream of the reactor 12. The gas exchanger 52 may be configured to discharge carbon dioxide mixed in the culture medium and to mix a predetermined gas component (for example, nitrogen N₂: 75%, oxygen O₂: 20%, and/or carbon dioxide CO₂: 5%) with the culture medium. An EC waste liquid circuit 48 may be configured to discharge a culture medium to the waste liquid unit 20 may be connected to the EC circulation circuit 44a downstream of the reactor 12. The EC supply circuit 44b may include an EC supply pump 30d configured to allow liquid to flow from the culture medium delivery route 40 to the EC circulation circuit 44a.

Although not illustrated, the plurality of medical bags 18 (e.g., cell solution bag 18A, cleaning solution bag 18B, and stripping solution bag 18C) may be connected to the IC supply circuit 42b upstream of the IC supply pump 30b or the EC supply circuit 44b upstream of the EC supply pump 30d via the plurality of tubes 22 in addition to the culture medium reservoir 14. In at least one example embodiment, one or more of the medical bags 18 may be replaced with a collection bag or the like using, for example, a sterile connecting device that sterilizes and bonds the bag depending on the intended use.

The sampling device 60 may be connected to the EC circulation circuit 44a at a position (e.g., between the reactor 12 and the EC waste liquid circuit 48) near the downstream side (of the reactor 12 (e.g., near the second EC terminal 36d). Therefore, one end of a sample outflow channel 54 through which a culture medium as a liquid sample flows out may be connected to the EC circulation circuit 44a. A culturing-device-side connector 56 may be provided at the other end of the sample outflow channel 54. The culturing-device-side connector 56 may be mutually connectable to a sampling-device-side connector 132 of the sampling device 60. The culturing device 11 may include a plurality of sample outflow channels 54 (culturing-device-side connectors 56) according to the number of the reactors 12 installed. In this case, a plurality of sample introduction channels 130 (sampling-device-side connectors 132) of the sampling device 60 may be provided according to the number of sample outflow channels 54.

An aseptic filter 58 may be provided at an intermediate position of the sample outflow channel 54. The aseptic filter 58 may be selected to maintain an aseptic state of the culture medium circulating through the culturing device 11 (EC circulation circuit 44a). In the sampling device 60, the sample outflow channel 54 may be connected to the IC circulation circuit 42a on the downstream side of the reactor 12 (e.g., near the second IC terminal 36b).

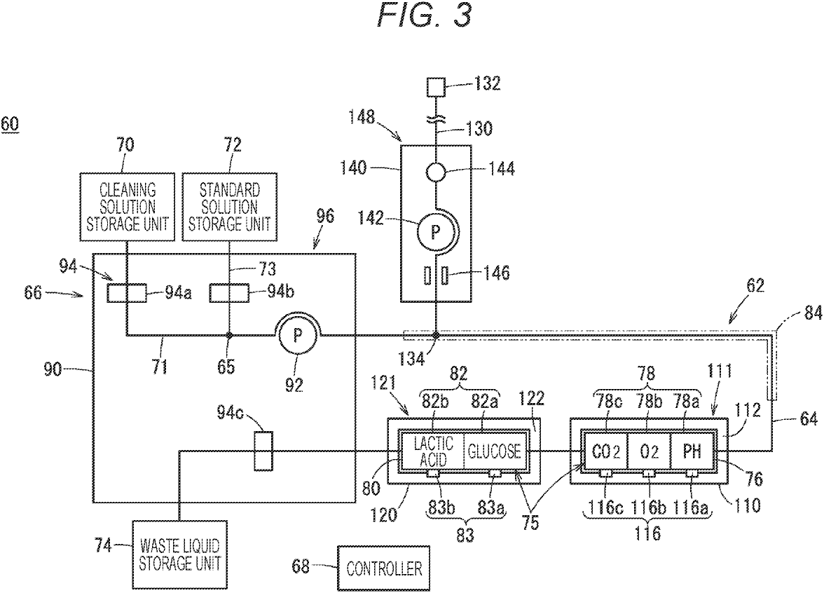

As illustrated in FIG. 3, the sampling device 60 may be configured to collect a sample of a culture medium from one or more culturing devices 11 to detect components contained in the sample, and to measure amounts (concentrations) of the components. The sampling device 60 may include a sampling kit 62 having a sampling channel 64 through which a sample is collected, a plurality of mechanisms 66 in which the sampling kit 62 is detachably set, and a controller 68 that controls operations of the plurality of mechanisms 66. In at least one example embodiment, the sampling kit 62 may be a disposable product that is used and thrown away, and the plurality of mechanisms 66 may be a reusable product.

In addition to the sampling channel 64, the sampling kit 62 may include a cleaning solution storage unit 70, a standard solution storage unit 72, a waste liquid storage unit 74, and/or a detection unit 75. The detection unit 75 may include a first detection unit 76 and a second detection unit 80. The sampling channel 64 may include a flexible tube having an appropriate thickness by which the sample can pass therethrough. The cleaning solution storage unit 70 may be connected to a branch point 65 to which one end of the sampling channel 64. For example, the cleaning solution storage unit 70 may be connected to the branch point 65 via a cleaning solution branch path 71. The standard solution storage unit 72 may be connected to the branch point 65 via a standard solution branch path 73. The other end of the sampling channel 64 may be connected to the waste liquid storage unit 74.

The cleaning solution storage unit 70 and the standard solution storage unit 72 may include a soft resin material that is formed into a bag shape (e.g., medical bag). The soft resin material may include, for example, polyvinyl chloride or polyolefin. The cleaning solution storage unit 70 and the standard solution storage unit 72 are not particularly limited as long as they can store liquid. In at least one example embodiment, the waste liquid storage unit 74 may share a tank with the waste liquid unit 20 of the culturing device 11, but it is not limited thereto, and a medical bag or the like may be applied.

The cleaning solution storage unit 70 may be configured to store a cleaning solution. The cleaning solution is not particularly limited. The cleaning solution as included in the cleaning solution storage unit 70 may be the same as or different form the cleaning solution as included in the cleaning solution bag 18B of the culturing device. For example, the cleaning solution as included in the cleaning solution storage unit 70 may include a buffer solution, a physiological saline solution, or the like described.

The standard solution storage unit 72 may be configured to store a standard solution. The standard solution may include a liquid for calibrating the first detection unit 76 and/or the second detection unit 80. The pH value, glucose value (glucose concentration), and lactic acid value (lactic acid concentration) of the standard solution liquid may be set to prescribed values. The sampling device 60 may include two or more standard solution storage units 72 that are configured to store standard solutions having different prescribed values and to perform two-point calibration for the first detection unit 76 and the second detection unit 80 by supplying two or more types of standard solutions at different timings.

The first detection unit 76 and the second detection unit 80 may be provided in series and separated from each other at an intermediate position of the sampling channel 64. Although illustrated as including the first detection unit 76 and the second detection unit 80, in other embodiments, the detection unit 75 may have a structure in which the first detection unit 76 and the second detection unit 80 are integrated or a structure divided into three or more units.

In at least one example embodiment, the first detection unit 76 may include tubular member having multiple first elements 78 that are configured to come in contact with (in liquid contact with) the sample in a flow path in the sampling channel 64. The multiple first elements 78 may include a pH chip 78a configured to measure the pH in the sample, an O₂ chip 78b configured to measure the O₂ concentration in the sample, and a CO₂ chip 78c configured to measure the CO₂ concentration in the sample. The pH chip 78a may be configured to change colors when reacted with H⁺ and/or OH⁻. The O₂ chip 78b may be configured to change colors when react with O₂. The CO₂ chip 78c may be configured to change colors when reacted with CO₂.

In at least one example embodiment, the second detection unit 80 may include a tubular member having multiple second elements 82 that are configured to come in contact with (in liquid contact with) the sample in the flow path in the sampling channel 64. The second detection unit 80 may be provided downstream of the first detection unit 76. For example, the second detection unit 80 may be disposed between the first detection unit 76 and the waste liquid storage unit 74. The multiple second elements 82 may include biosensors that are configured to react enzymes with a circulating sample and to detect a current change or the like. For example, the multiple second elements 82 may include a glucose chip 82a that is configured to measure the glucose concentration in the sample and/or a lactic acid chip 82b that is configured to measure the lactic acid concentration in the sample.

The glucose chip 82a may be electrically connected to a glucose terminal 83a protruding to the outside of the tubular member of the second detection unit 80. The lactic acid chip 82b may be electrically connected to a lactic acid terminal 83b protruding to the outside of the tubular member second detection unit 80. The glucose terminal 83a and/or the lactic acid terminal 83b may be integrated as an electrode terminal 83 with an insulating material therebetween.

The sampling kit 62 may include a connection part 84 to which one or more sample introduction channels 130 can be connected. For example, the connection part 84 may be disposed between the branch point 65 of the sampling channel 64 and the first detection unit 76. In FIG. 3, the connection part 84 is indicated as a range surrounded by a two-dot chain line. The connection part 84 may include a member obtained by integrally molding a plurality of branch ports each having a valve (not illustrated) that closes when the sample introduction channel 130 is not attached and opens as the sample introduction channel 130 is attached.. Alternatively, a port to which the sample introduction channel 130 can be connected with the sampling channel 64 being kept aseptic can be applied to the connection part 84.

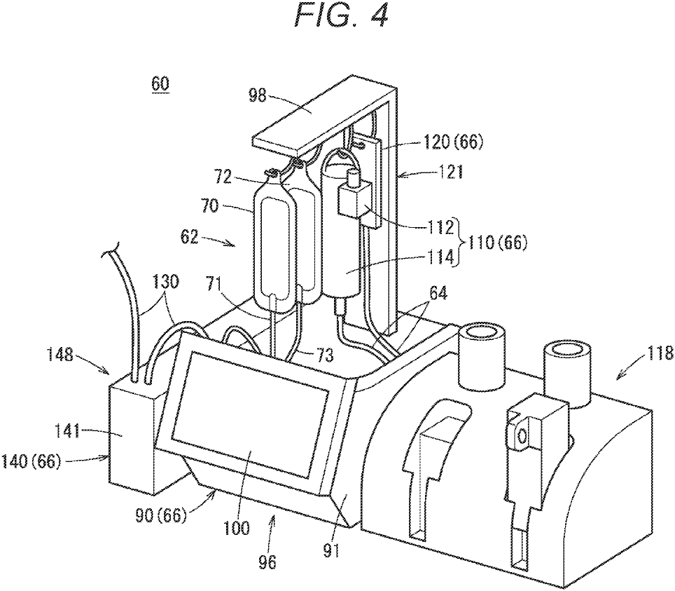

At least a part of the sampling kit 62 may be set in a main mechanism 90, which is one of the plurality of mechanisms 66 as illustrated in FIGS. 3 and 4. The main mechanism 90 may include a housing 91 that houses a motor and/or an actuator (which are not illustrated). The housing 91 may include, on the housing surface thereof, a groove (not illustrated) that holds a part of the cleaning solution branch path 71, a part of the standard solution branch path 73, a predetermined region from the branch point 65 of the sampling channel 64 to the connection part 84, and/or a predetermined region of the sampling channel 64 from the second detection unit 80 to the waste liquid storage unit 74. The main mechanism 90 may also include a main-mechanism-side pump 92 (e.g., a first pump) and a plurality of clamps 94 that are configured to open and close flow paths in the respective channels (e.g., tubes). Although not illustrated, the controller 68 that is configured to control the sampling device 60 may also be provided in the main mechanism 90.

The sampling channel 64 may extend between the branch point 65 and the connection part 84 and may be disposed in the main-mechanism-side pump 92. The main-mechanism-side pump 92 may have a circular wound portion around which the sampling channel 64 can be wound so as to wrap around. The circular wound portion may be configured to rotate so as to apply a peristaltic action on the wrapping sampling channel 64 (tube) thereby allowing an internal fluid (liquid, air, etc.) to flow.

The multiple clamps 94 may include a cleaning solution clamp 94a in which the cleaning solution branch path 71 may be disposed, a standard solution clamp 94b in which the standard solution branch path 73 may be disposed, and a waste liquid clamp 94c in which the sampling channel 64 between the second detection unit 80 and the waste liquid storage unit 74 may be disposed. The cleaning solution clamp 94a may be configured to switch between allowing the cleaning solution in the cleaning solution storage unit 70 to flow and interrupting the flow of the cleaning solution by opening and closing the cleaning solution branch path 71 under the control of the controller 68. The standard solution clamp 94b may be configured to switch between allowing the standard solution in the standard solution storage unit 72 to flow and interrupting the flow of the standard solution by opening and closing the standard solution branch path 73 under the control of the controller 68. The waste liquid clamp 94c may be configured to switch between inflow and interruption of inflow of the liquid in the waste liquid storage unit 74 by opening and closing the sampling channel 64 under the control of the controller 68.

The sampling kit 62 may be set in the main mechanism 90 by which a main unit 96 of the sampling device 60 is constructed. The main unit 96 may be configured such that the sampling kit 62 (e.g., part of the sampling channel 64 including the region from the cleaning solution storage unit 70 to the downstream side of the main-mechanism-side pump 92), the main-mechanism-side pump 92, and the multiple clamps 94 can be integrally handled.

As illustrated in FIG. 4, the main unit 96 (main mechanism 90) may include a stand 98 configured to suspend the cleaning solution storage unit 70 and the standard solution storage unit 72 on the top of the housing 91. The main unit 96 may include a door-like monitor 100 on the front surface of the housing 91. The groove, the main-mechanism-side pump 92, and the multiple clamps 94 may be provided on the housing surface on the back side of the monitor 100. The user may set the sampling kit 62 after opening the monitor 100, and may then close the monitor 100. This may prevent the sampling channel 64 from falling off. The connection part 84 (sampling channel 64) exposed from the main unit 96 may be placed on the upper surface of the housing 91. The cleaning solution branch path 71 exposed from the housing 91 may extend to the cleaning solution storage unit 70, and the standard solution branch path 73 similarly exposed from the housing 91 may extend to the standard solution storage unit 72.

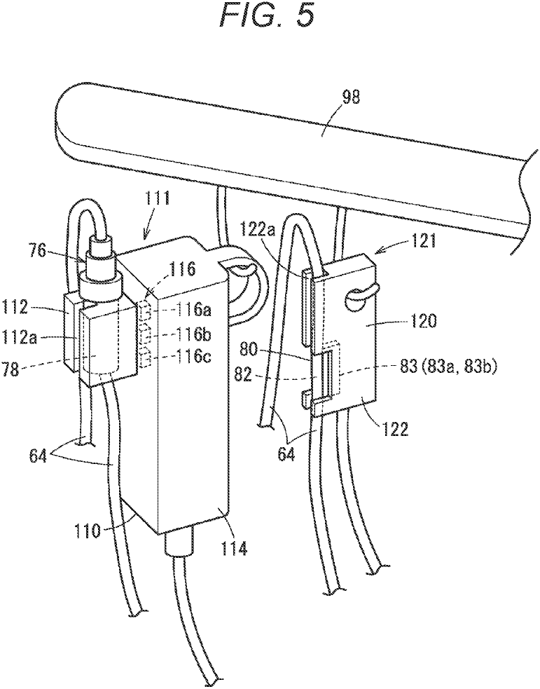

As illustrated in FIGS. 3 and 5, the first detection unit 76 of the sampling kit 62 may be set in a first measuring instrument 110 which is one of the plurality of mechanisms 66. The first measuring instrument 110 may include a holder 112 having, for example, a square tube shape and configured to accommodate the plurality of first elements 78. The first measuring instrument 110 may also include a cylindrical measurement body 114 to which the holder 112 is fixed and that is configured to optically measure the plurality of first elements 78. The holder 112 may be formed to have light shielding properties and may include a recess 112a for housing and holding the first detection unit 76 from the side.

The measurement body 114 may include optical detectors 116 that are arranged to face the plurality of first elements 78 (e.g., pH chip 78a, O₂ chip 78b, and/or CO₂ chip 78c) with the first detection unit 76 being held by the holder 112. That is, the plurality of optical detectors 116 may include a pH detector 116a, an O₂ detector 116b, and/or a CO₂ detector 116c. Under the control of the controller 68, each optical detector 116 may be configured to emit measurement light having a wavelength corresponding to the characteristics of the corresponding first element 78 and to receive excitation light generated from the first element 78 by excitation. Thus, each optical detector 116 may be configured to transmit a detection signal based on the degree of coloration of the corresponding first element 78 to the controller 68.

The first measuring instrument 110 may be accommodated in a calibration device 118 in order to perform calibration when the first detection unit 76 is not set. The calibration device 118 may be configured to bubble a predetermined gas component into a standard solution (not shown) and to calibrate the relationship between the luminosity detected by each optical detector 116 of the set first measuring instrument 110 and the measured values of pH and/or O₂ and/or CO₂ (concentrations) which are subjects for detection.

The second detection unit 80 of the sampling kit 62 may be set in a second measuring instrument 120, which is one of the plurality of mechanisms 66. The second measuring instrument 120 may include a plate-like case 122 capable of accommodating the plurality of electrode terminals 83 protruding from the plurality of second detection units 80. The case 122 may have a recess 122a for housing and holding the second detection units 80 from the side and a port (not illustrated) into which the electrode terminals 83 may be inserted.

The second measuring instrument 120 may include an enzyme detector (not illustrated) electrically connected to the glucose terminal 83a and/or the lactic acid terminal 83b in a state where the second detection unit 80 is held in the case 122. The enzyme detector may be configured to detect a current value from each of the glucose chip 82a and/or the lactic acid chip 82b and to transmit a detection signal based on the current value to the controller 68.

In the sampling device 60, a first sensor unit 111 may be constructed by setting the first detection unit 76 in the first measuring instrument 110, and a second sensor unit 121 may be constructed by setting the second detection unit 80 in the second measuring instrument 120. The first sensor unit 111 and the second sensor unit 121 may be provided to be separated from each other, whereby different detection methods are possible and the respective sensor units can be easily set.

In order to introduce a sample to be measured by the first sensor unit 111 and the second sensor unit 121, the sample introduction channel 130 may be connected to the connection part 84 of the sampling kit 62 (sampling channel 64) as illustrated in FIG. 3. Similar to the sampling channel 64, the sample introduction channel 130 may be constituted by a flexible tube having an appropriate thickness by which the sample can pass therethrough.

The sample introduction channel 130 may have, at one end, a sampling-device-side connector 132 to be connected to the culturing-device-side connector 56 (see also FIG. 2). A plug (not illustrated) attachable to and detachable from the connection part 84 may be provided at the other end of the sample introduction channel 130. Hereinafter, a portion where the sample introduction channel 130 is connected to the sampling channel 64 may be referred to as a connection point 134.

A part of the sample introduction channel 130 may be detachably set to an introduction mechanism 140, which is one of the plurality of mechanisms 66. The introduction mechanism 140 may include a rectangular housing 141 that accommodates a motor (not illustrated) and may be configured such that the sample introduction channel 130 passes through the housing 141 (see also FIG. 4). The housing 141 may be disposed at a position near the flow path control mechanism 24 of the culturing device 11. The introduction mechanism 140 may include, within the housing 141, an introduction-mechanism-side pump 142 (second pump), a pressure sensor 144 that is configured to detect the pressure in the flow path of the sample introduction channel 130, and/or a bubble sensor 146 that is configured to detect air bubbles in the flow path of the sample introduction channel 130.

The introduction-mechanism-side pump 142 may include a circular wound portion around which the sample introduction channel 130 can be wound so as to wrap around. The circular wound portion may be configured to rotate so as to apply a peristaltic action on the wrapping sample introduction channel 130 (tube) thereby allowing an internal fluid (liquid, air, etc.) to flow. The introduction mechanism 140 including the introduction-mechanism-side pump 142 may be preferably set near the connection point 134.

The pressure sensor 144 may be configured to detect an internal pressure between the sampling-device-side connector 132 and the introduction-mechanism-side pump 142 (for example, on the upstream side with respect to the introduction-mechanism-side pump 142) in the sample introduction channel 130. The detection result detected by the pressure sensor 144 may be wirelessly transmitted to the controller 68. In order to enhance the pressure detection accuracy of the pressure sensor 144, the place where the pressure sensor 144 is to be disposed in the sample introduction channel 130 may have an appropriate shape (e.g., cylindrical shape, disk shape, or the like having, for example, a larger diameter than other portions).

In at least one example embodiment, the bubble sensor 146 may be provided between the introduction-mechanism-side pump 142 and the plug (e.g., connection point 134) in the sample introduction channel 130 and may be configured to detect air bubbles in the sample introduction channel 130. The detection result detected by the bubble sensor 146 may be wirelessly transmitted to the controller 68. In other example embodiments, the bubble sensor 146 may be provided upstream of the introduction-mechanism-side pump 142.

The sample introduction channel 130 may be set in the introduction mechanism 140 by which an introduction unit 148 of the sampling device 60 is constructed. The introduction unit 148 may be configured such that a part of the sample introduction channel 130, the introduction-mechanism-side pump 142, the pressure sensor 144, and/or the bubble sensor 146 can be integrally handled. The sample introduction channel 130 may extend from the introduction unit 148 and may be connected to the connection part 84 on the main unit 96.

The main-mechanism-side pump 92 may be disposed in the sampling channel 64 between the branch point 65 and the connection point 134 (e.g., (downstream side of the cleaning solution storage unit 70 and the standard solution storage unit 72) of the sample introduction channel 130. The cleaning solution clamp 94a may be disposed in the cleaning solution branch path 71 between the cleaning solution storage unit 70 and the branch point 65. The standard solution clamp 94b may be disposed in the standard solution branch path 73 between the standard solution storage unit 72 and the branch point 65.

The introduction-mechanism-side pump 142 may be disposed in the sample introduction channel 130 between the connection part 84 (connection point 134) of the sampling channel 64 downstream of the main-mechanism-side pump 92 and the sampling-device-side connector 132. The length of the sampling channel 64 from the connection part 84 to the main-mechanism-side pump 92 may be shorter than the length of the sample introduction channel 130 from the connection part 84 to the introduction-mechanism-side pump 142.

The controller 68 (control unit) may be a computer including one or more processors (not illustrated), a memory, an input/output interface, and/or an electronic circuit. The controller 68 may be configured to control the entire sampling device 60 by executing program(s) stored in the memory. The controller 68 may be connected to the main unit 96, the first sensor unit 111, the second sensor unit 121, and/or the introduction unit 148 via a wireless or wired communication module so as to be able to communicate information. The controller 68 may be configured to control the operations of the main-mechanism-side pump 92, the plurality of clamps 94, and/or the introduction-mechanism-side pump 142. The controller 68 may be configured to receive detection signals of the first measuring instrument 110, the second measuring instrument 120, the pressure sensor 144, and the bubble sensor 146 and to perform various types of processing. The controller 68 may be a control device integrated with the control circuit 32 of the culturing device 11.

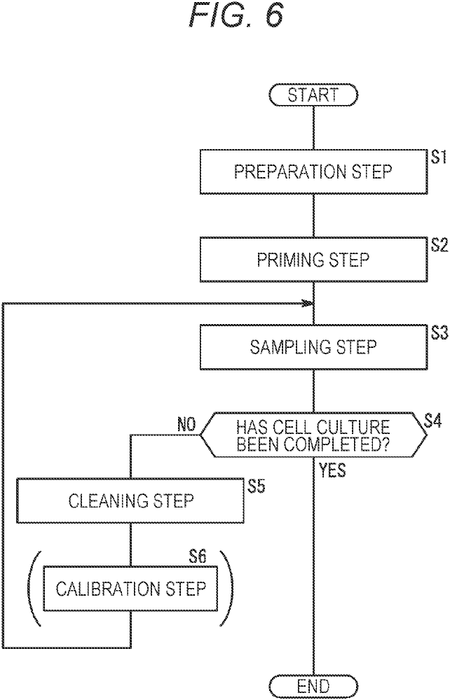

The sampling method that uses, for example, the sampling device 60, is illustrated in FIG. 6. The sampling method may include a preparation step, a priming step, a sampling step, a cleaning step, and/or a calibration step which may be sequentially performed.

First, in the preparation step (step S1), the user of the cell culturing system 10 may set (e.g., attaches) the sampling kit 62 to the main mechanism 90 to form the main unit 96 as illustrated in FIGS. 3 to 5. The user may set the first detection unit 76 exposed from the housing 91 in the first measuring instrument 110 to construct the first sensor unit 111. The user may set the second detection unit 80, which are similarly exposed in the second measuring instrument 120, to construct the second sensor unit 121. The first sensor unit 111 and the second sensor unit 121 may be suspended from the stand 98.

The user may set the sample introduction channel 130 in the introduction mechanism 140 to form the introduction unit 148. The user may connect the sampling-device-side connector 132 of the sample introduction channel 130 exposed from the introduction unit 148 to the culturing-device-side connector 56. The user may connect the plug of the sample introduction channel 130 to the connection part 84.

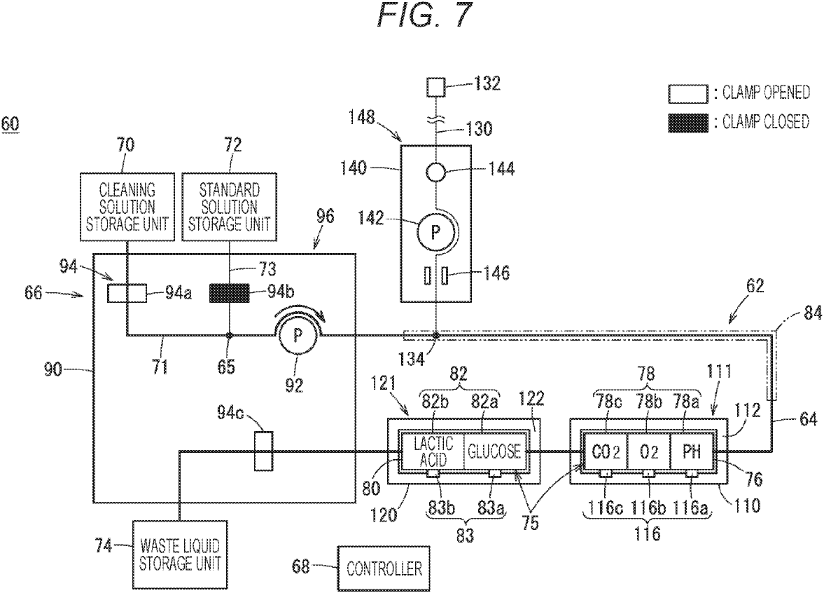

In the priming step (step S2 in FIG. 6), the controller 68 may be configured to open the cleaning solution clamp 94a and the waste liquid clamp 94c and to close the standard solution clamp 94b as illustrated in FIG. 7. The controller 68 may be configured to rotates the main-mechanism-side pump 92. Due to the rotation of the main-mechanism-side pump 92, a negative pressure may be applied to the cleaning solution branch path 71, so that the cleaning solution is supplied from the cleaning solution storage unit 70. The cleaning solution that has passed through the cleaning solution branch path 71 and the branch point 65 may pass through the main-mechanism-side pump 92 in the sampling channel 64. The cleaning solution may sequentially flow through the connection part 84, the first detection unit 76, and/or the second detection unit 80 and may be discharged to the waste liquid storage unit 74 due to a positive pressure being applied from the main-mechanism-side pump 92. In the priming step, the introduction-mechanism-side pump 142 may stop rotating by which the inflow of the cleaning solution into the sample introduction channel 130 can be avoided.

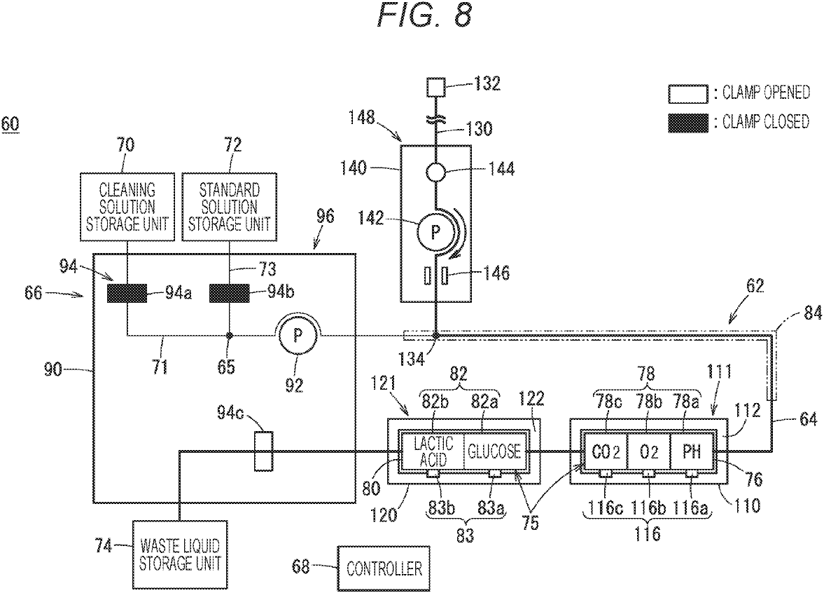

The sampling device 60 may be configured to collect a sample from the culturing device 11 in the next sampling step (step S3 in FIG. 6). The controller 68 may be configured to close the cleaning solution clamp 94a and the standard solution clamp 94b and to open the waste liquid clamp 94c as shown in FIG. 8. The controller 68 may also be configured to rotate the introduction-mechanism-side pump 142 while stopping the rotation of the main-mechanism-side pump 92. Due to the rotation of the introduction-mechanism-side pump 142, a negative pressure may be applied to the sample introduction channel 130 on the upstream side of the introduction-mechanism-side pump 142 whereby the sample is introduced from the culturing device 11.

The sample drawn from the culturing device 11 may pass through the aseptic filter 58 in the sample outflow channel 54 (see FIG. 2) and may be guided to the sample introduction channel 130. When flowing through the sample introduction channel 130 and passing through the introduction-mechanism-side pump 142, the sample sequentially may flow through the connection part 84 (connection point 134), the first detection unit 76, and/or the second detection unit 80 and may be discharged to the waste liquid storage unit 74 due to a positive pressure being applied from the introduction-mechanism-side pump 142.

When the sample passes, the plurality of first elements 78 (e.g., pH chip 78a, O₂ chip 78b, and/or CO₂ chip 78c) of the first detection unit 76 may come into contact with the sample and may be colored according to the pH and/or the contents of O₂ and/or CO₂. The first measuring instrument 110 may be configured to optically measure each of the first elements 78 and to transmit the detection result to the controller 68. The controller 68 that has received the detection result may be configured to perform appropriate processing to display the measured values (e.g., pH value, O₂ concentration, and/or CO₂ concentration) on the monitor 100.

Similarly, when the sample passes, the plurality of second elements 82 (glucose chip 82a and/or lactic acid chip 82b) of the second detection unit 80 may come into contact with the sample, and the second measuring instrument 120 may be configured to detect current values corresponding to the contents of glucose and/or lactic acid. The second measuring instrument 120 may be configured to transmit each detection result to the controller 68. The controller 68 that has received the detection result may be configured to perform appropriate processing to display the measured values (glucose concentration and/or lactic acid concentration) on the monitor 100.

In the sampling step, a negative pressure may be applied to the sample introduction channel 130 upstream of the introduction-mechanism-side pump 142, whereas a positive pressure may be applied to the sample introduction channel 130 and the sampling channel 64 downstream of the introduction-mechanism-side pump 142. Therefore, when the sampling device 60 finishes the sampling step and stops the rotation of the introduction-mechanism-side pump 142, the pressure in the sample introduction channel 130 may become uniform, so that the pressure difference between the negative pressure and the positive pressure gradually disappears. Therefore, the negative pressure in the sample introduction channel 130 may be eliminated in a short time.

In the sampling step, the pressure sensor 144 of the introduction unit 148 may be configured to detect the internal pressure of the sample introduction channel 130 on the upstream side of the introduction-mechanism-side pump 142 and to transmit the detection result to the controller 68. The controller 68 may be configured to recognize how much negative pressure may be applied based on the detection result of the pressure sensor 144. For example, when the internal pressure of the sample introduction channel 130 is equal to or higher than a predetermined pressure threshold, the controller 68 may be configured to decrease the rotation speed or stops the rotation of the introduction-mechanism-side pump 142 and to rotate the introduction-mechanism-side pump 142 after a lapse of a certain period of time (after the negative pressure decreases).

In the sampling step, the bubble sensor 146 of the introduction unit 148 may be configured to detect air bubbles in the sample introduction channel 130 and to transmit the detection result to the controller 68. Air bubbles may be generated by collecting gas (e.g., N₂, O₂, and/or CO₂) in the sample by negative pressure or the like. Therefore, when air bubbles are detected by the bubble sensor 146, the controller 68 may be configured to decrease the rotation speed or stops the rotation of the introduction-mechanism-side pump 142 in the same manner as described above.

After the sampling step, the controller 68 may be configured to determine whether or not the cell culture of the culturing device 11 is completed (step S4). When the cell culture is not completed (step S4: NO), the cleaning step (step S5) may not be performed. In the cleaning step, the controller 68 may be configured to supply the cleaning solution in the cleaning solution storage unit 70 to the sampling channel 64, as in the priming step illustrated in FIG. 7. As a result, the sample attached to the plurality of first elements 78 (e.g., pH chip 78a, O₂ chip 78b, and/or CO₂ chip 78c) and the plurality of second elements 82 (glucose chip 82a and/or lactic acid chip 82b) may be removed by the cleaning solution.

At the start of the cleaning step, the introduction-mechanism-side pump 142 may stops, so that the sample introduction channel 130 is always shut off from the sampling channel 64 as described above. Therefore, even if the inside of the sample introduction channel 130 has a negative pressure, entry of the cleaning solution flowing through the sampling channel 64 into the sample introduction channel 130 can be prevented.

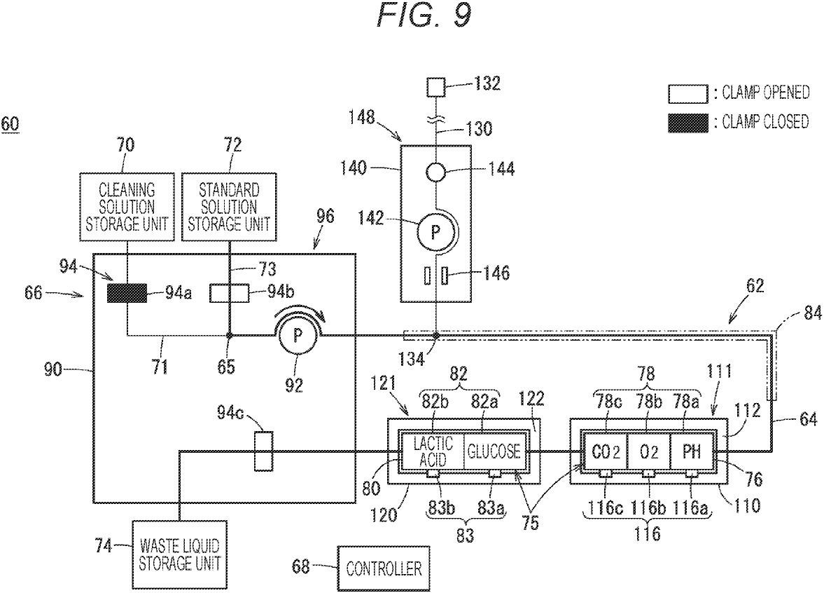

The sampling device 60 may be configured to perform a calibration step (step S6 in FIG. 6) as necessary. In the calibration step, the controller 68 may be configured to rotate the main-mechanism-side pump 92 with the standard solution clamp 94b and the waste liquid clamp 94c being opened and the cleaning solution clamp 94a being closed as illustrated in FIG. 9. The standard solution in the standard solution storage unit 72 may be guided from the standard solution branch path 73 to the sampling channel 64 by the action of the main-mechanism-side pump 92. The standard solution may pass through the main-mechanism-side pump 92 in the sampling channel 64, may sequentially flows through the connection part 84, the first detection unit 76, and/or the second detection unit 80, and may be discharged to the waste liquid storage unit 74.

The second sensor unit 121 may be configured to measure the glucose concentration and/or the lactic acid concentration in the standard solution and to transmit the measurement results to the controller 68 and/or the second measuring instrument 120. The controller 68 and/or the second measuring instrument 120 may be configured to calibrate the second measuring instrument 120 on the basis of the measurement result of the second sensor unit 121. The first sensor unit 111 (first measuring instrument 110) may be set in the calibration device 118 to measure the standard solution, pH, O₂ concentration, and/or CO₂ concentration in the calibration device 118 and to transmit the measurement results to the controller 68 or the first measuring instrument 110. The controller 68 and/or the first measuring instrument 110 may be configured to perform calibration of each of the pH detector 116a, the O₂ detector 116b, and/or the CO₂ detector 116c on the basis of the measurement results.

With renewed reference to FIG. 6, when the cleaning step (or the calibration step) is completed, the controller 68 may return to step S3 and may performs the subsequent steps. On the other hand, when determining in step S4 that the cell culture is completed (step S4: YES), the controller 68 may be configured to end the operation flow of the sampling device 60.

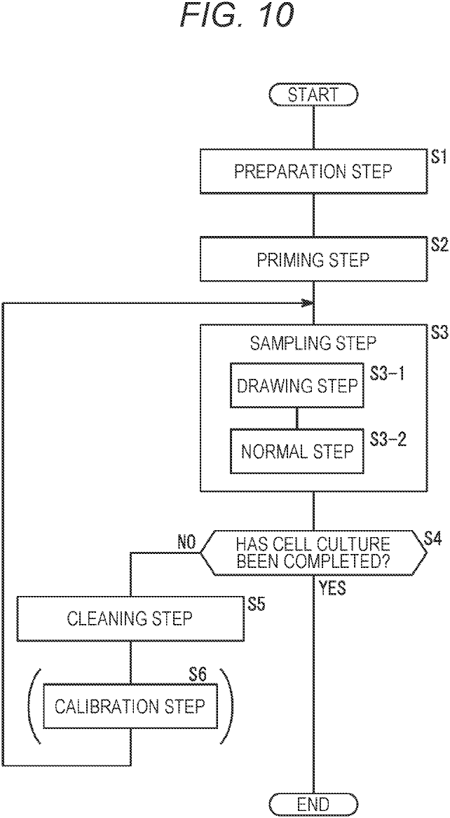

Note that the sampling method performed by the sampling device 60 is not limited to the above method, and various methods can be adopted. For example, as illustrated in FIG. 10, the sampling device 60 may be configured to perform a drawing step (step S3-1) for drawing the sample to the upstream side (to the main-mechanism-side pump 92) a little with respect to the connection point 134 under the rotation of the main-mechanism-side pump 92 at the start of the sampling step.

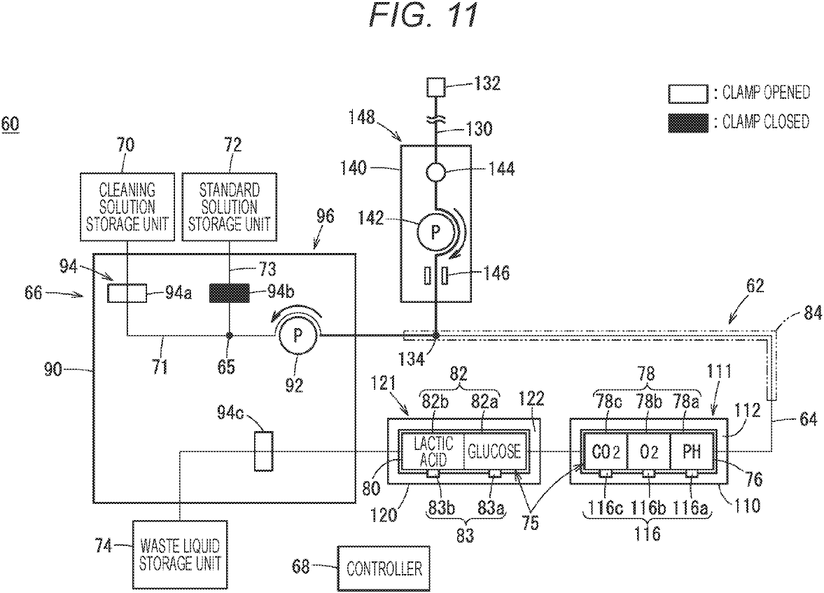

In the drawing step, the controller 68 may be configured to rotate the introduction-mechanism-side pump 142 to introduce the sample and to rotate the main-mechanism-side pump 92 in a rotation direction opposite to the rotation direction during the priming step, as illustrated in FIG. 11. The controller 68 may be configured to open the cleaning solution clamp 94a. Thus, the sample introduced from the sample introduction channel 130 may move to the sampling channel 64 located closer to the main-mechanism-side pump 92 with respect to the connection point 134. Although not particularly limited, the amount of movement of the sample may be set so that the sample stays in a region in front of the sampling channel 64 wound around the main-mechanism-side pump 92.

After executing the drawing step for a short time, the controller 68 may be configured to stop the rotation of the main-mechanism-side pump 92 and to close the cleaning solution clamp 94a. Thereafter, the controller 68 may be configured to perform the normal step in FIG. 10 (step S3-2: sampling step illustrated in FIG. 8). That is, the controller 68 may be configured to rotate the introduction-mechanism-side pump 142 to introduce the sample from the sample introduction channel 130 to the sampling channel 64 and to sequentially delivers the sample to the first detection unit 76 and the second detection unit 80.

After the drawing step is performed, the sample may be located in the sampling channel 64 on the upstream side (main-mechanism-side pump 92 side) with respect to the connection point 134. Therefore, during the sampling step, the sample and the cleaning solution may be prevented from being mixed at the connection point 134. Thus, the sampling device 60 can further improve the detection accuracy of the pH, O₂ concentration, CO₂ concentration, glucose concentration, and/or lactic acid concentration of the sample.

The present disclosure is not limited to the abovementioned embodiment and various modifications are possible without departing from the spirit of the invention. For example, although in the described embodiments the sampling device 60 includes the pressure sensor 144 and the bubble sensor 146 in the introduction unit 148, in other embodiments, the introduction unit 148 may not include these sensors. Alternatively, or additionally, the introduction unit 148 may include only one of the pressure sensor 144 and the bubble sensor 146.

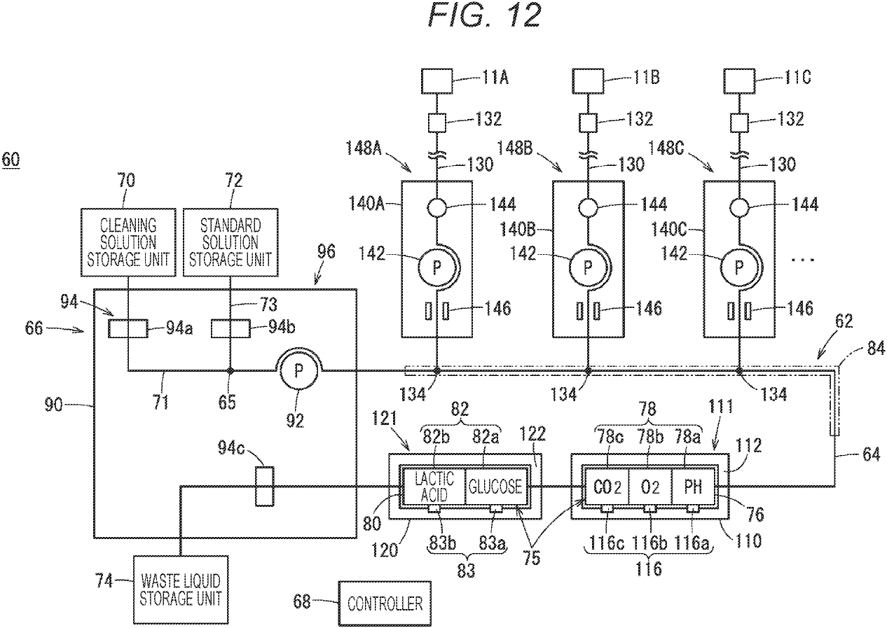

In at least one example embodiment, as illustrated in FIG. 12, the sampling device 60 may be configured such that the sample introduction channels 130 are respectively connected to a plurality of different culturing devices 11 (e.g., culturing device 11A, culturing device 11B, and/or culturing device 11C), and the plurality of sample introduction channels 130 are connected to the connection part 84. The sample introduction channels 130 may be set in a plurality of introduction mechanisms 140 (e.g., introduction mechanism 140A, introduction mechanism 140B, and/or introduction mechanism 140C ...), respectively, whereby a plurality of introduction units 148 (e.g., introduction unit 148A, introduction unit 148B, and/or introduction unit 148C) are constructed. Each introduction unit 148 may include an introduction-mechanism-side pump 142, a pressure sensor 144, and/or a bubble sensor 146.

The controller 68 may be configured to perform the cleaning step after performing the sampling step on the culturing device 11A (introduction unit 148A) and then to perform the sampling step on the culturing device 11B (introduction unit 148B). Similarly, the cleaning step and the sampling step may be repeated by the number of introduction units 148 connected to the sampling channel 64. As described above, even in the configuration in which the sample of each culturing device 11 is collected, the sampling device 60 may sequentially measure the target component contained in each sample and an amount (concentration) of the component.

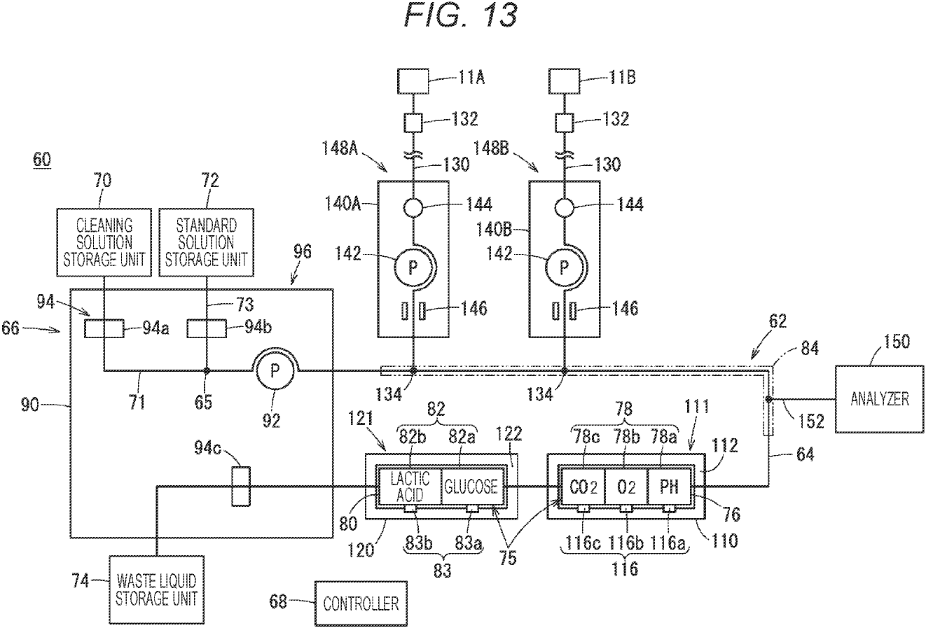

In addition, as illustrated in FIG. 13, an analysis channel 152 connected to an analyzer 150 that is configured to analyze the sample may be connected to the connection part 84 of the sampling channel 64 of the sampling device 60. The analyzer 150 is not particularly limited. In certain embodiments, the analyzer 150 may include high-performance liquid chromatography (HPLC) for separating a target component (e.g., O₂, CO₂, glucose, and/or lactic acid) from a sample and performing qualitative and quantitative analysis.

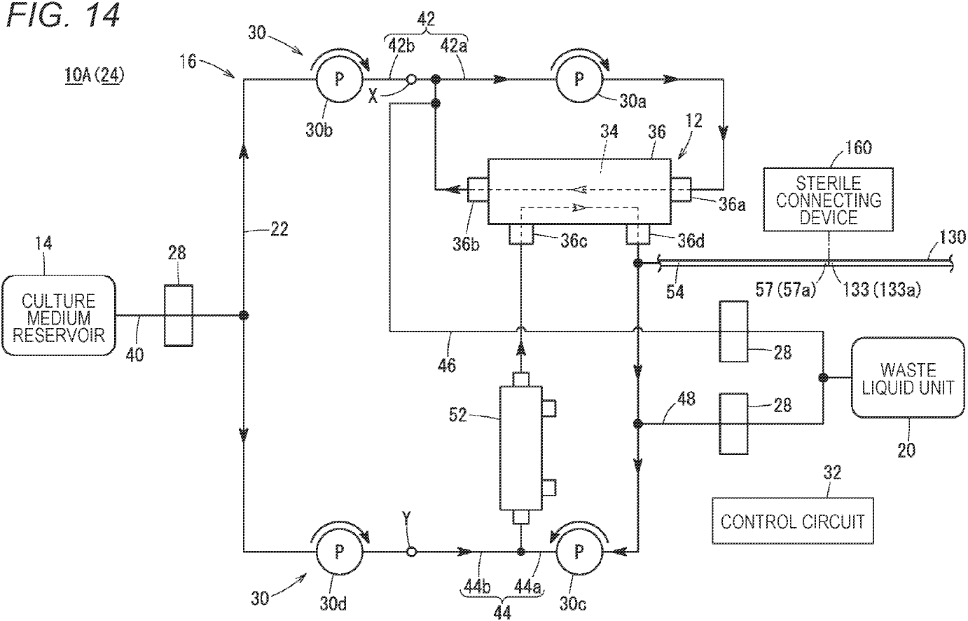

In at least one example embodiment, as illustrated in FIG. 14, a cell culturing system 10A (the culturing device 11 and the sampling device 60) may not include the aseptic filter 58. For example, the cell culturing system 10A may ensure the sterility of the culturing device 11 by aseptically connecting a joint portion 57 of the culturing device 11 and a joint portion 133 of the sampling device 60 by a sterile connecting device 160. The joint portions 57 and 133 are not particularly limited as long as they can ensure sterility. For example, in at least one example embodiment, a closed tube having a closed distal end can be applied. That is, the sample outflow channel 54 of the culturing device 11 may include a culturing-device-side connection end 57a as the joint portion 57, and the sample introduction channel 130 of the sampling device 60 may include a sampling-device-side connection end 133a as the joint portion 133.

In at least one example embodiment, the cell culturing system 10A may include a plurality of joint portions 57 and joint portions 133 according to the number of the reactors 12 installed. That is, the cell culturing system 10A may include a plurality of sample outflow channels 54 (culturing-device-side connection ends 57a) and a plurality of sample introduction channels 130 (sampling-device-side connection ends 133a) and the respective connection ends may be connected by the sterile connecting device 160.

In at least one example embodiment, the present disclosure provides a sampling device 60 for collecting a sample in a liquid form from a culturing device 11 that cultures cells. The sampling device 60 may including a sampling channel 64 through which the sample flows; a detection unit 75 provided in the sampling channel 64 so as to come into contact with the sample; a cleaning solution storage unit 70 that is connected to the sampling channel 64 upstream of the detection unit 75 and configured to store a cleaning solution; and a sample introduction channel 130 connected to the culturing device 11 and connected to the sampling channel 64 between the detection unit 75 and the cleaning solution storage unit 70. The sample introduction channel 130 may enable the introduction of the sample from the culturing device 11 and into the sampling channel 64. The sampling device 60 may include a first pump (e.g., main-mechanism-side pump 92) and a second pump (e.g., introduction-mechanism-side pump 142). The first pump may be provided in the sampling channel 64 between the cleaning solution storage unit 70 and the sample introduction channel 130 and configured to allow the cleaning solution to flow to the detection unit 75. The second pump may be provided in the sample introduction channel 130 and may be configured to allow the sample to flow to the detection unit 75 from the sample introduction channel 130.

With the above configuration, the sampling device 60 can clean the detection unit 75 by supplying the cleaning solution to the detection unit 75 using the first pump (e.g., main-mechanism-side pump 92) and can measure the sample by supplying the sample to the detection unit 75 using the second pump (e.g., introduction-mechanism-side pump 142). The sampling device 60 can effectively prevent entry of the cleaning solution from the sampling channel 64 into the sample introduction channel 130 by shutting off the sample introduction channel 130 using the second pump.

An aseptic filter 58 that can prevent entry of bacteria into the culturing device 11 may be provided between the culturing device 11 and the second pump (e.g., introduction-mechanism-side pump 142). Even if a flowing amount of the sample is reduced by the aseptic filter 58 and a negative pressure is generated in the sample introduction channel 130, the sampling device 60 can shut the sample introduction channel 130 off from the sampling channel 64 using the second pump. Thus, even if the inside of the sample introduction channel 130 has a negative pressure, entry of the cleaning solution flowing through the sampling channel 64 into the sample introduction channel 130 can be prevented.

The sampling device may include a control unit (e.g., controller 68) that is configured to control the operation of each of the first pump (e.g., main-mechanism-side pump 92) and the second pump (e.g., introduction-mechanism-side pump 142). For example, the control unit may be configured such that the first pump allows the cleaning solution to flow and the second pump allows the sample to flow at different times. With such a configuration, the sampling device 60 may repeat the cleaning step and the sampling step, while ensuring the detection accuracy of the sample in the detection unit 75.

The sampling device may include a standard solution storage unit 72 connected to the sampling channel 64 upstream of the first pump (e.g., main-mechanism-side pump 92) and configured to store a standard solution for calibrating the detection unit 75. The sampling channel 64 may be connected to a cleaning solution branch path 71 that is connected to the cleaning solution storage unit 70 and a standard solution branch path 73 that is connected to the standard solution storage unit 72 on an upstream side with respect to the first pump. The sampling device may include a cleaning solution clamp 94a that opens and closes the cleaning solution branch path 71 and a standard solution clamp 94b that opens and closes the standard solution branch path 73. The control unit (e.g., controller 68) may open one of the cleaning solution clamp 94a and the standard solution clamp 94b and close the other of the cleaning solution clamp 94a and the standard solution claim 94b as the first pump operates. The sampling device 60 may calibrate the detection unit 75 by allowing the standard solution to flow as necessary.

At least part of the sampling channel 64 including, for example, a region from the cleaning solution storage unit 70 to a downstream side of the first pump (e.g., main-mechanism-side pump 92) and the first pump may be formed as a main unit 96 that is capable of being integrally handled. With this configuration, the user may easily handle the sampling device 60.

At least part of the sample introduction channel 130 and the second pump (e.g., introduction-mechanism-side pump 142) may be formed as an introduction unit 148 that is capable of being integrally handled. With this configuration, the user may easily handle the introduction unit 148 and can connect the sample introduction channel 130 extending from the introduction unit 148 to the sampling channel 64.

The introduction unit 148 may include at least one of a pressure sensor 144 and a bubble sensor 146. The pressure sensor 144 may be configured to detect an internal pressure of the sample introduction channel 130 upstream of the second pump (e.g., introduction-mechanism-side pump 142). The bubble sensor 146 may be configured to detect air bubbles in the sample introduction channel 130. With this configuration, the sampling device 60 may monitor the negative pressure of the sample introduction channel 130 and can take an appropriate measure. Further, due to the sensor(s) being provided in the introduction unit 148, the sensor(s) may be easy to handle.

The sampling channel 64 may include a connection part 84 to which the sample introduction channels 130 and/or an analysis channel 152 are connected. The sample introduction channels 130 may extend from a plurality of the introduction units 148. The analysis channel 152 may be in communicate with an analyzer 150 configured to analyze the sample. The connection part 84 may be provided on an exposed portion of the main unit 96 downstream of the first pump (e.g., main-mechanism-side pump 92). With this configuration, the sampling device 60 may measure the samples of the plurality of culturing devices 11 using one device.

The detection unit 75 may include one or more elements (e.g., first element 78 and/or second element 82) disposed in the sampling channel 64. The one or more elements may be integrally mountable in a measuring instrument (including, for example, first measuring instrument 110 and/or second measuring instrument 120) which is provided separately from the main unit 96. With this configuration, the user may set the detection unit 75 (including, for example, the first sensor unit 111 and/or second sensor unit 121) more easily.

In at least one example embodiment, the present disclosure provides a cell culturing system 10 that includes a culturing unit (e.g., culturing device 11) for culturing a cell. The cell culturing system 10 may also include a sampling channel 64 through which a liquid sample as collected from the culturing unit flows; a detection unit 75 provided in the sampling channel 64 so as to come into contact with the sample; a cleaning solution storage unit 70 that is connected to the sampling channel 64 upstream of the detection unit 75 and is configured to store a cleaning solution; and a sample introduction channel 130 connected to the culturing unit and connected to the sampling channel 64 between the detection unit 75 and the cleaning solution storage unit 70. The sample introduction channel 130 may enable introduction of the sample from the culturing unit and into the sampling channel 64. The cell culturing system 10 may include a first pump (e.g., main-mechanism-side pump 92) and a second pump (e.g., introduction-mechanism-side pump 142). The first pump may be provided in the sampling channel 64 between the cleaning solution storage unit 70 and the sample introduction channel 130 and may be configured to allow the cleaning solution to flow to the detection unit 75. The second pump may be provided in the sample introduction channel 130 and configured to allow the sample to flow to the detection unit 75 from the sample introduction channel 130.

1. A sampling device for receiving a sample from a cell culturing device, the sampling device comprising:
a sampling channel through which the sample flows;
a detection unit disposed in the sampling channel so as to come into contact with the sample;
a cleaning solution storage unit in fluid communication with the sampling channel upstream of the detection unit and configured to store a cleaning solution;
a sample introduction channel in fluid communication with the sampling channel between the detection unit and the cleaning solution storage unit and also in fluid communication with the cell culturing device, the sample introduction channel enabling movement of the sample from the culturing device and into the sampling channel;
a first pump disposed in the sampling channel between the cleaning solution storage unit and the sample introduction channel and configured to allow the cleaning solution to flow from the cleaning solution storage unit to the detection unit; and
a second pump disposed in the sample introduction channel and configured to allow the sample to flow from the sample introduction channel to the detection unit.

2. The sampling device of claim 1, wherein the sampling device further includes:
an aseptic filter disposed between the cell culturing device and the second pump, the aseptic filter configured to prevent entry of bacteria into the cell culturing device.

3. The sampling device of claim 1, wherein the sampling device further includes:
a control unit configured to control operation of the first pump and the second pump.

4. The sampling device of claim 3, wherein the control unit is configured to initiate the first pump and the second pump at different times.

5. The sampling device of claim 3, wherein the sampling device further includes:
a standard solution storage unit in fluid communication with the sampling channel upstream of the first pump and configured to store a standard solution for calibrating the detection unit.

6. The sampling device of claim 5, wherein the sampling channel is in fluid communication with a cleaning solution branch path that is in fluid communication with the cleaning solution storage unit, the sampling channel is also in fluid communication with a standard solution branch path that is in fluid communication with the standard solution storage unit.

7. The sampling device of claim 6, wherein the sampling device further includes:
a cleaning solution clamp that is configured to open and close the cleaning solution branch path; and
a standard solution clamp that is configured to open and close the standard solution branch path.

8. The sampling device of claim 7, wherein the control unit is configured to open one of the cleaning solution clamp and the standard solution clamp and to close the other of the cleaning solution clamp and the standard solution clamp as the first pump operates.

9. The sampling device of claim 1, wherein at least a part of the sampling channel and also the first pump are formed as a main unit that is configured to be integrally handled.

10. The sampling device of claim 9, wherein the part of the sampling channel includes a region of the sampling channel between the cleaning solution storage unit and the first pump.

11. The sampling device of claim 9, wherein the sampling channel includes a connection part configured to receive at least a portion of the sample introduction channel, wherein the connection part is provided on a portion exposed from the main unit downstream of the first pump.

12. The sampling device of claim 9, wherein the detection unit includes one or more elements, and the one or more elements are integrally mounted in a measuring instrument that is provided separately from the main unit.

13. The sampling device of claim 1, wherein at least a part of the sample introduction channel and also the second pump are formed as an introduction unit that is configured to be integrally handled.

14. The sampling device of claim 13, wherein the introduction unit includes at least one of:
a pressure sensor that is configured to detect an internal pressure of the sample introduction channel; and
a bubble sensor that is configured to detect an air bubble in the sample introduction channel.

15. The sampling device of claim 14, wherein the pressure sensor is disposed in the sample introduction channel upstream of the second pump.

16. The sampling device of claim 14, wherein the bubble sensor is disposed in the sample introduction channel downstream of the second pump.

17. A cell culturing system comprising:
a culturing unit configured to culture cells;
a sampling channel configured to receive a sample from the culturing unit;
a detection unit disposed in the sampling channel so as to come into contact with the sample;
a cleaning solution storage unit in fluid communication with the sampling channel upstream of the detection unit and configured to store a cleaning solution;
a sample introduction channel in fluid communication with the sampling channel between the detection unit and the cleaning solution storage unit and also in fluid communication with the culturing unit, the sample introduction channel enabling movement of the sample from the culturing unit and into the sampling channel;
a first pump disposed in the sampling channel between the cleaning solution storage unit and the sample introduction channel and configured to allow the cleaning solution to flow from the cleaning solution storage unit to the detection unit; and
a second pump disposed in the sample introduction channel and configured to allow the sample to flow to the detection unit from the sample introduction channel.

18. The cell culturing system of claim 17, wherein the cell culturing system further includes:
an aseptic filter disposed between the cell culturing device and the second pump, the aseptic filter configured to prevent entry of bacteria into the cell culturing device.

19. The cell culturing system of claim 17, wherein the sampling device further includes:
a control unit configured to control operation of the first pump and the second pump including initiating the first pump and the second pump at different times;
a standard solution storage unit in fluid communication with the sampling channel upstream of the first pump and configured to store a standard solution for calibrating the detection unit.

20. The cell culturing system of claim 19, wherein the sampling channel is in fluid communication with a cleaning solution branch path that is in fluid communication with the cleaning solution storage unit, the sampling channel is also in fluid communication with a standard solution branch path that is in fluid communication with the standard solution storage unit;
wherein the sampling device further includes a cleaning solution clamp that is configured to open and close the cleaning solution branch path, and a standard solution clamp that is configured to open and close the standard solution branch path; and
wherein the control unit is configured to open one of the cleaning solution clamp and the standard solution clamp and to close the other of the cleaning solution clamp and the standard solution clamp as the first pump operates.
